(19) United States
(12) Patent Application Publication
(10) Pub. No.: US 2022/0233733 A1
(43) Pub. Date: Jul. 28, 2022

(54) DISINFECTION SYSTEM CAPABLE OF DIRECTLY GENERATING OZONE WATER IN WATER PIPE WITH GENERATOR THAT CAN BE REPLACED QUICKLY

(71) Applicant: Yantai United Ozonetec Corporation, Yantai (CN)

(72) Inventors: MING-YUNG HSU, Yantai (CN); WEI-CHUN HSU, Yantai (CN)

(21) Appl. No.: 17/161,648

(22) Filed: Jan. 28, 2021

(51) Int. Cl. A61L 2/18; H01R 39/64; A61L 2/26; C01B 13/11; C02F 1/467; C02F 1/461

(52) U.S. Cl. CPC A61L 2/183; H01R 39/64; A61L 2/26; A61L 2101/02; C02F 1/4672; C02F 1/46109; C01B 13/115

(57) ABSTRACT

A disinfection system includes an ozone water generator and a top seat. A base of the top seat has a top seat entrance and a top seat exit. A middle portion of an underside of the base is formed with an opening having a top seat inlet and a top seat outlet to communicate with the top seat entrance and the top seat exit. A power interface is provided on an outer cover of the top seat. Generator claws are interlocked with top seat claws. A generator mouth at a middle portion of the upper end of the ozone water generator is inserted into the opening. The generator mouth is formed with a generator inlet and a generator outlet to communicate with the top seat inlet and the top seat outlet. An anode and a cathode of the ozone water generator are movably connected to the generator control board through wires. The disinfection system is able to avoid unnecessary waste and improve convenience of use.

### FIELD OF THE INVENTION

The present invention relates to a disinfection system, and more particularly to a disinfection system capable of directly generating ozone water in a water pipe with a generator that can be replaced quickly.

### BACKGROUND OF THE INVENTION

A disinfection system capable of directly generating ozone water generally uses a coated titanium anode material. This material has a relatively short service life. If the whole disinfection system is discarded, it will cause a great waste. Therefore, it is necessary to design a disinfection product with a generator that can be replaced quickly.

### SUMMARY OF THE INVENTION

The primary object of the present invention is to overcome the above-mentioned shortcomings of the prior art and to provide a disinfection system capable of directly generating ozone water in a water pipe with a generator that can be replaced quickly.

In order to achieve the above object, a disinfection system is provided. The disinfection system comprises an ozone water generator and a top seat movably connected to the ozone water generator. The top seat includes a base and an outer cover. The base has a top seat entrance and a top seat exit. A middle portion of an underside of the base is formed with an opening. The opening at the underside of the base is formed with a top seat inlet communicating with the top seat entrance and a top seat outlet communicating with the top seat exit. A generator control board is provided inside the outer cover. A power interface is provided on the outer cover. Two sides of a lower end of the outer cover are provided with top seat claws. Two sides of an upper end of the ozone water generator are provided with generator claws matching the top seat pawls. The generator claws are interlocked with the top seat claws. A middle portion of the upper end of the ozone water generator is provided with a generator mouth. The generator mouth is inserted into the opening. The generator mouth is formed with a generator inlet and a generator outlet. The generator inlet communicates with the top seat inlet. The generator outlet communicates with the top seat outlet. An anode and a cathode of the ozone water generator are movably connected to the generator control board.

Preferably, the outer cover is provided with a quick connection socket. The quick connection socket is connected to the generator control board through a wire. The anode and the cathode of the ozone water generator are connected to a quick connection plug through wires. The quick connection plug is connected to the quick connection socket.

Alternatively, two sides of a lower portion of the opening at the underside of base are provided with an anode conductive slip ring and a cathode conductive slip ring, respectively. The anode conductive slip ring and the cathode conductive slip ring are respectively connected to the generator control board through wires. An upper portion of the ozone water generator is provided with an anode conductive reed and a cathode conductive reed. The anode conductive reed and the cathode conductive reed are connected to the anode and the cathode of the ozone water generator. The anode conductive slip ring and the cathode conductive slip ring are connected with the anode conductive reed and the cathode conductive reed, respectively.

Preferably, an outer wall of the generator mouth is provided with an O-shaped sealing ring, and the O-shaped sealing ring is located between the generator mouth and the opening.

The beneficial effect of the disinfection system provided by the present invention is to avoid unnecessary waste and improve the convenience for use.

### BRIEF DESCRIPTION OF THE DRAWINGS

FIG. 1 is a structural schematic view of a first embodiment of the present invention; and

FIG. 2 is a structural schematic view of a second embodiment of the present invention.

### DETAILED DESCRIPTION OF THE PREFERRED EMBODIMENTS

Embodiments of the present invention will now be described, by way of example only, with reference to the accompanying drawings.

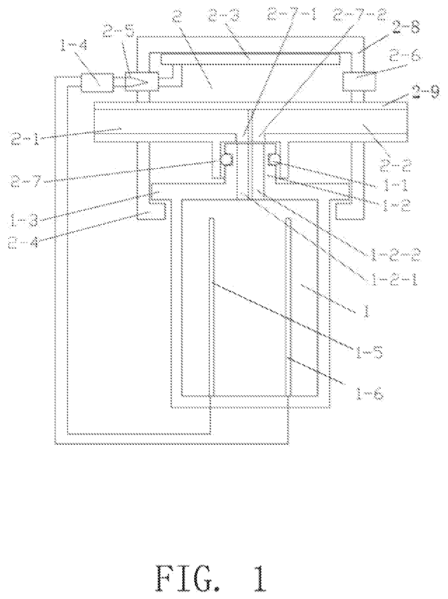

FIG. 1 illustrates a first embodiment of a disinfection system capable of directly generating ozone water in a water pipe with a generator that can be replaced quickly. The disinfection system comprises an ozone water generator 1 and a top seat 2 movably connected to the ozone water generator 1. The top seat 2 includes a base 2-9 and an outer cover 2-8. The base 2-9 has a top seat entrance 2-1 and a top seat exit 2-2. The middle portion of the underside of the base 2-9 is formed with an opening 2-7. The opening 2-7 at the underside of the base 2-9 is formed with a top seat inlet 2-7-1 communicating with the top seat entrance 2-1 and a top seat outlet 2-7-2 communicating with the top seat exit 2-2. A generator control board 2-3 is provided inside the outer cover 2-8. A power interface 2-6 and a quick connection socket 2-5 are provided on the outer cover 2-8. The quick connection socket 2-5 is connected to the generator control board 2-3 through a wire. Two sides of the lower end of the outer cover 2-8 are provided with top seat claws 2-4. Two sides of the upper end of the ozone water generator 1 are provided with generator claws 1-3 matching the top seat pawls 2-4. The generator claws 1-3 are interlocked with the top seat claws 2-4. The middle portion of the upper end of the ozone water generator 1 is provided with a generator mouth 1-2. The generator mouth 1-2 is inserted into the opening 2-7. The outer wall of the generator mouth 1-2 is provided with an O-shaped sealing ring 1-1. The O-shaped sealing ring 1-1 is located between the generator mouth 1-2 and the opening 2-7 for sealing. The generator mouth 1-2 is formed with a generator inlet 1-2-1 and a generator outlet 1-2-2. The generator inlet 1-2-1 communicates with the top seat inlet 2-7-1. The generator outlet 1-2-2 communicates with the top seat outlet 2-7-2. An anode 1-5 and a cathode 1-6 of the ozone water generator 1 are connected to a quick connection plug 1-4 through wires. The quick connection plug 1-4 is connected to the quick connection socket 2-5.

When in use, water flows through the top seat entrance 2-1, the top seat inlet 2-7-1 and the generator inlet 1-2-1 to enter the generator. The generator control board 2-3 is powered through the power interface 2-6. The generator control board 2-3 supplies a constant current power supply to the generator through the quick-connect socket 2-5, the quick-connect plug 1-4 and the wires. The voltage range of the power supply is 3.5-12 V to start the generator. Water is electrolyzed under the action of an electric field, and oxygen ions generate ozone and oxygen microbubbles under the action of the anode catalyst. Ozone microbubbles quickly dissolve into water to directly generate ozone water. The ozone water flows out through the generator outlet 1-2-2, the top seat outlet 2-7-2 and the top seat exit 2-2 for removing the biofilm on the pipe wall and sterilizing the water in the pipe.

When the generator needs to be replaced, the quick connection plug 1-4 is first unplugged, and the generator is rotated an angle to disengage the top seat claws 2-4 from the generator claws 1-3, and the generator is pulled out. A new generator is installed in reverse order, so as to complete the replacement of the generator.

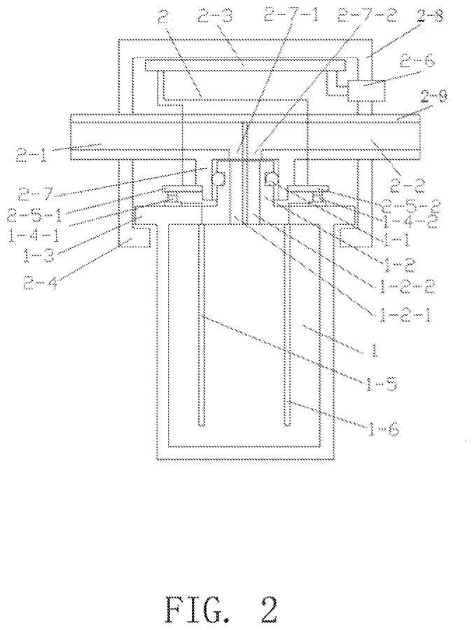

FIG. 2 illustrates a second embodiment of a disinfection system capable of directly generating ozone water in a water pipe with a generator that can be replaced quickly. The difference between the second embodiment and the first embodiment is that the circuit connection of the generator is different, without the quick connection plug 1-4 and the quick connection socket 2-5. Two sides of a lower portion of the opening 2-7 at the underside of base 2-9 are provided with an anode conductive slip ring 2-5-1 and a cathode conductive slip ring 2-5-2, respectively. The anode conductive slip ring 2-5-1 and the cathode conductive slip ring 2-5-2 are respectively connected to the generator control board 2-3 through wires. An upper portion of the ozone water generator 1 is provided with an anode conductive reed 1-4-1 and a cathode conductive reed 1-4-2. The anode conductive reed 1-4-1 and the cathode conductive reed 1-4-2 are connected to the anode 1-5 and the cathode 1-6 of the ozone water generator 1. The anode conductive slip ring 2-5-1 and the cathode conductive slip ring 2-5-2 are connected with the anode conductive reed 1-4-1 and the cathode conductive reed 1-4-2, respectively.

When the generator needs to be replaced, the generator is rotated an angle to disengage the top seat claws 2-4 from the generator claws 1-3, and the generator is pulled out. The anode conductive slip ring 2-5-1 and the cathode conductive slip ring 2-5-2 are separated from the anode conductive reed 1-4-1 and the cathode conductive reed 1-4-2, respectively. A new generator is installed in reverse order, so as to complete the replacement of the generator.

Although particular embodiments of the present invention have been described in detail for purposes of illustration, various modifications and enhancements may be made without departing from the spirit and scope of the present invention. Accordingly, the present invention is not to be limited except as by the appended claims.

What is claimed is:

1. A disinfection system, comprising an ozone water generator (1) and a top seat (2) movably connected to the ozone water generator (1), the top seat (2) including a base (2-9) and an outer cover (2-8), the base (2-9) having a top seat entrance (2-1) and a top seat exit (2-2), a middle portion of an underside of the base (2-9) being formed with an opening (2-7), the opening (2-7) at the underside of the base (2-9) being formed with a top seat inlet (2-7-1) communicating with the top seat entrance (2-1) and a top seat outlet (2-7-2) communicating with the top seat exit (2-2), a generator control board (2-3) being provided inside the outer cover (2-8), a power interface (2-6) being provided on the outer cover (2-8); two sides of a lower end of the outer cover (2-8) being provided with top seat claws (2-4); two sides of an upper end of the ozone water generator (1) being provided with generator claws (1-3) matching the top seat pawls (2-4), the generator claws (1-3) being interlocked with the top seat claws (2-4); a middle portion of the upper end of the ozone water generator (1) being provided with a generator mouth (1-2), the generator mouth (1-2) being inserted into the opening (2-7), the generator mouth (1-2) being formed with a generator inlet (1-2-1) and a generator outlet (1-2-2), the generator inlet (1-2-1) communicating with the top seat inlet (2-7-1), the generator outlet (1-2-2) communicating with the top seat outlet (2-7-2); an anode (1-5) and a cathode (1-6) of the ozone water generator (1) being movably connected to the generator control board (2-3).

2. The disinfection system as claimed in claim 1, wherein the outer cover (2-8) is provided with a quick connection socket (2-5), the quick connection socket (2-5) is connected to the generator control board (2-3) through a wire; the anode (1-5) and the cathode (1-6) of the ozone water generator (1) are connected to a quick connection plug (1-4) through wires, and the quick connection plug (1-4) is connected to the quick connection socket (2-5).

3. The disinfection system as claimed in claim 1, wherein two sides of a lower portion of the opening (2-7) at the underside of base (2-9) are provided with an anode conductive slip ring (2-5-1) and a cathode conductive slip ring (2-5-2) respectively, the anode conductive slip ring (2-5-1) and the cathode conductive slip ring (2-5-2) are respectively connected to the generator control board (2-3) through wires; an upper portion of the ozone water generator (1) is provided with an anode conductive reed (1-4-1) and a cathode conductive reed (1-4-2), the anode conductive reed (1-4-1) and the cathode conductive reed (1-4-2) are connected to the anode (1-5) and the cathode (1-6) of the ozone water generator (1), the anode conductive slip ring (2-5-1) and the cathode conductive slip ring (2-5-2) are connected with the anode conductive reed (1-4-1) and the cathode conductive reed (1-4-2), respectively.

4. The disinfection system as claimed in claim 1, wherein an outer wall of the generator mouth (1-2) is provided with an O-shaped sealing ring (1-1), and the O-shaped sealing ring (1-1) is located between the generator mouth (1-2) and the opening (2-7).
